(19) United States
(12) Patent Application Publication
(10) Pub. No.: US 2019/0180851 A1
(43) Pub. Date: Jun. 13, 2019

(54) System and method for predicting non-adherence risk based on socio-economic determinates of health

(71) Applicant: KONINKLIJKE PHILIPS N.V., EINDHOVEN (NL)

(72) Inventors: Eran SIMHON, Boston, MA (US); Reza SHARIFI SEDEH, Malden, MA (US)

(21) Appl. No.: 16/203,822

(22) Filed: Nov. 29, 2018

Related U.S. Application Data
(60) Provisional application No. 62/596,946, filed on Dec. 11, 2017.

(51) Int. Cl. G16H 10/60; G16H 50/30; G06N 20/00

(52) U.S. Cl. CPC G16H 10/60; G06N 20/00; G16H 50/30

(57) ABSTRACT

In one embodiment, a computer system and method is disclosed for providing a social determinates of health model which correlates socio-economic factors with the non-adherence risk of an individual to a proscribed task such as care plan compliance, medication dosing, physical therapy or exercise routine, office visit, or test appointment.

### CROSS-REFERENCE TO PRIOR APPLICATIONS

This application claims the benefit of U.S. Provisional Application No. 62/596,946, filed on 11 Dec. 2017. This application is hereby incorporated by reference herein.

### TECHNICAL FIELD

Various exemplary embodiments disclosed herein relate generally to computer systems for determining non-adherence risk, and more particularly to computer systems for providing a non-adherence risk score based on socio-economic determinates of health.

### BACKGROUND

The delivery of healthcare is evolving rapidly driven in no small part by the high cost of health care and an aging worldwide demographic with increased healthcare needs. Without systemic changes, there will be an increased percentage of the population without meaningful access to healthcare. Historically, patient care was performed on a case-by-case basis focused on episodic interventions by a care provider often at the request of the patient.

In addition, care providers may be generalists, or specialists with a particular field of expertise, and may lack expertise in every possible medical nuance of a given patient's total clinical condition. Therefore, these care providers may only take into account a subset of the issues the patient is struggling with based on their particular expertise and miss other important issues. This may result in the caregiver failing to address some critical health issues. Or even worse, in some instances, their guidance may even create, or exacerbate, the other health issues.

Further, caregivers often do not have immediate access to the patient's complete medical history. Patient records are often unavailable, incomplete, or the caregiver may simply not have sufficient time to review the records in the time allotted. Care providers often have to rely on their personal judgment to make the best care decision possible with limited information and limited time allotted during a private consultation. Such decisions could end up being biased, outdated, based on erroneous information or otherwise suboptimal.

Due to the issues noted above, this method of patient-initiated, episodic care management has been found to have several drawbacks. Patients left to manage their own care tend to postpone seeking medical counsel until late stages in their disease progression, thus increasing healthcare costs with a resulting decreased quality of life. This is in contrast with early interventions, which are known to decrease or eliminate negative health consequences at reduced cost.

In the fee-for-service system, the caregiver may have the wrong incentive. Since they are compensated based on services rendered and penalized for poor outcomes. Their financial motivation is aligned with maximizing the delivery of services, which may be at odds with the cost-effective delivery of care. And, if they happen to overlook something they could be held responsible so it is better to over prescribe diagnostics and therapy, even if the likely need is remote. And, in many instances, once the patient seeks medical counsel, they too are often unable or unmotivated to evaluate the counsel given. They are handicapped by their lack of medical expertise, and when they are covered by health insurance, they too have little motivation to control costs. So, when the care provider stands to profit and the patient has little or no ability to disagree, expensive interventions with remote therapeutic value may be pursued thus burdening the healthcare system and needlessly raising insurance costs.

To address the inefficiency of fee-for-service healthcare, government and private payers have sought to correct this situation and provide compensation to caregivers based on their performance rewarding superior outcomes and cost-efficiency otherwise known as value based care. While theoretically desirable, it has proven difficult to develop metrics, which create the right incentives. If the focus is solely on outcomes, then any intervention even with modest therapeutic benefit should be pursued. The obvious down side to this approach is, as with episodic care, this method will result in waste and extreme cost. On the other end of the spectrum, an incentive system could focus solely on costs. Taken to an extreme, cost savings can be realized by refusing even therapeutic services leading to poor outcomes. So, as there has been a migration from a fee-for-service model to these incentive-based systems, it has pitted patients and physicians against payers. Patients and physicians often accuse payers of putting profits ahead of delivering on their promise of providing health coverage. And, payers argue that patients seek costly interventions when the therapeutic value is suspect. Intellectually, the problem becomes one of optimization, the goal should be to optimize care to provide the highest quality at the lowest cost. However, given the issues noted above, identifying the optimized care has proven challenging especially when the goal is to develop an intervention for a particular patient with unique characteristics.

In an effort to address this problem, there has been a rise in evidence-based medicine to support the shift from the fee-for-service model to value based care. Rather than relying on the expertise of a singular caregiver, evidence-based medicine applies scientific principles to the delivery of care in order to optimize decision-making based on a review of longitudinal patient data to develop standards of care or best practices. In these models, metrics are developed to compensate the caregiver based on their performance and seeks to reward superior results relative to their peers.

While providing clear advantages, one challenge with evidence-based medicine has been the collection, storage, and analysis of large volumes of patient data in an effort to provide a customized care plan for patients or populations. To mitigate this issue, electronic medical records systems (EMRs) have been developed to assist with data management. These systems typically include the same information that would have been retained by individual caregivers across the healthcare network. Advantageously, EMRs provide a centralized repository for patient records including patient history, demographics, past visits, imaging, and the like. Collecting these records in a central repository facilitates caregiver collaboration and the longitudinal data can be used for better evidence-based care analytics thus providing a fuller picture of the patient's clinical picture.

The shift to evidence-based medicine and analysis of EMR data has been an improvement, but additional advances are desired. Disappointingly, it has been found that demographic and healthcare factors typically captured in EMRs are not as predicative of the patient's healthcare outcomes as one would expect and are, in fact, somewhat poor at predicting a patient's health status and likely outcome, thereby undermining the promise of evidence-based care as a means to achieve optimized healthcare. It has been found that equally, if not more predicative of a patient's health status and outcomes, are socio-economic factors such as economic, environmental, educational, social, and behavior factors. The data contained in current EMRs provides an imperfect picture of the patient and lacks any insight into these important socio-economic determinates of health.

One of the main issues leading to healthcare waste and inefficiency is non-adherence, which is becoming a bigger problem as healthcare is transitioning towards greater reliance on at-home and self-managed care. There are many benefits derived from at-home healthcare including the promise of reduced cost and better clinical outcomes. However, one problem that has emerged is that as more responsibility for managing an illness transfers from the care provider to the patient, it makes non-adherence more likely. Non-adherence can take many forms. It may result in missed doctor's appointments, exams, care plan compliance, medication adherence.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a system for producing an adjustable socio-economic adherence model, including: a database containing socio-economic data which classifies socio-economic factors into socio-economic categories and containing electronic medical record (EMR) data for a plurality of patients; a user interface displayed on a display device to a user, wherein the user interface is configured to: display socio-economic categories to the user; and receive user inputs selecting one or more socio-economic categories; a computing device configured to produce the adjustable socio-economic adherence model, wherein the computing device is configured to: extract training patient data from the EMR data including patient task adherence data; extract socio-economic training data for the patients from the socio-economic data based upon socio-economic data and the user selected socio-economic categories; and train the adjustable socio-economic model using the extracted patient data and extracted socio-economic training data, wherein the adjustable socio-economic model produces a non-adherence risk score related to the patient task.

Various embodiments are described, wherein the socio-economic categories include an environmental category, an educational category, an economic category, a social category, and a behavior category.

Various embodiments are described, wherein the user interface is further configured to: present individuals with upcoming tasks; and receive a user selection of an individual and a task assigned to the individual; and the computing device is further configured to calculate a non-adherence risk score for the selected individual and selected task using the adjustable socio-economic model.

Various embodiments are described, wherein the system includes a table that correlates tasks with a task importance index, the non-adherence risk score is further based on the task importance index, the computing device is further configured to train the adjustable socio-economic model to calculate a root-cause related to the non-adherence risk, and the user interface is further configured to display the task non-adherence score, the task importance index, and the root-cause related to the non-adherence for a selected individual.

Various embodiments are described, further including the system includes a table that correlates tasks with a task importance index and wherein the non-adherence risk score is further based on the task importance index.

Various embodiments are described, further including the computing device is further configured to train the adjustable socio-economic model to calculate a root-cause related to the non-adherence risk.

Various embodiments are described, further including training the adjustable socio-economic model comprises training a sub-model for each socio-economic category and wherein the non-adherence risk score is calculated as a weighted sum of each of the sub-models.

Various embodiments are described, wherein the computing device is further configured to calculate a measure of the adjustable socio-economic model's predictive quality, and the user interface is further configured to display the adjustable socio-economic model's predictive quality to the user.

Further various embodiments relate to a method of producing an adjustable socio-economic adherence model, including: displaying, by a user interface, socio-economic categories to a user; receiving, by the user interface, user inputs selecting one or more socio-economic categories; extracting, by a computing device, training patient data from emergency electronic medical record (EMR) data in a database including patient task adherence data; extracting, by the computing device, socio-economic training data for the patients from socio-economic data in the database based upon the user selected socio-economic categories; and training, by the computing device, the adjustable socio-economic model using the extracted patient data and extracted socio-economic training data, wherein the adjustable socio-economic model produces a non-adherence risk score related to the patient task.

Various embodiments are described, wherein the socio-economic categories include an environmental category, an educational category, an economic category, a social category, and a behavior category.

Various embodiments are described, further including presenting, by the user interface, individuals with upcoming tasks; receiving, by the user interface, a user selection of an individual and a task assigned to the individual; and calculating, by the computing device, a non-adherence risk score for the selected individual and selected task using the adjustable socio-economic model.

Various embodiments are described, further including: training, by the computing device, the adjustable socio-economic model to calculate a root-cause related to the non-adherence risk; and displaying, by the user interface, a task non-adherence score, a task importance index, and the root-cause related to the non-adherence for a selected individual, wherein the system includes a table that correlates tasks with a task importance index, and wherein the non-adherence risk score is further based on the task importance index,

Various embodiments are described, wherein the system includes a table that correlates tasks with a task importance index and wherein the non-adherence risk score is further based on the task importance index.

Various embodiments are described, further including training, by the computing device, the adjustable socio-economic model to calculate a root-cause related to the non-adherence risk.

Various embodiments are described, wherein training the adjustable socio-economic model further comprises training a sub-model for each socio-economic category and wherein the non-adherence risk score is calculated as a weighted sum of each of the sub-models.

Various embodiments are described, further including: calculating, by the computing device, a measure of the adjustable socio-economic model's predictive quality, and displaying, by the user interface, the adjustable socio-economic model's predictive quality to the user.

Further various embodiments relate to a non-transitory machine-readable storage medium encoded with instructions for producing an adjustable socio-economic adherence model, including: instructions for displaying, by a user interface, socio-economic categories to a user; instructions for receiving, by the user interface, user inputs selecting one or more socio-economic categories; instructions for extracting, by a computing device, training patient data from emergency electronic medical record (EMR) data in a database including patient task adherence data; instructions for extracting, by the computing device, socio-economic training data for the patients from socio-economic data in the database based upon the user selected socio-economic categories; and instructions for training, by the computing device, the adjustable socio-economic model using the extracted patient data and extracted socio-economic training data, wherein the adjustable socio-economic model produces a non-adherence risk score related to the patient task.

Various embodiments are described, wherein the socio-economic categories include an environmental category, an educational category, an economic category, a social category, and a behavior category.

Various embodiments are described, further including: instructions for presenting, by the user interface, individuals with upcoming tasks; instructions for receiving, by the user interface, a user selection of an individual and a task assigned to the individual; and instructions for calculating, by the computing device, a non-adherence risk score for the selected individual and selected task using the adjustable socio-economic model.

Various embodiments are described, further including: instructions for training, by the computing device, the adjustable socio-economic model to calculate a root-cause related to the non-adherence risk; and instructions for displaying, by the user interface, a task non-adherence score, a task importance index, and the root-cause related to the non-adherence for a selected individual, wherein the system includes a table that correlates tasks with a task importance index, and wherein the non-adherence risk score is further based on the task importance index,

Various embodiments are described, wherein the system includes a table that correlates tasks with a task importance index and wherein the non-adherence risk score is further based on the task importance index.

Various embodiments are described, further including instructions for training, by the computing device, the adjustable socio-economic model to calculate a root-cause related to the non-adherence risk.

Various embodiments are described, wherein instructions for training the adjustable socio-economic model further comprises instructions for training a sub-model for each socio-economic category and wherein the non-adherence risk score is calculated as a weighted sum of each of the sub-models.

Various embodiments are described, further including: instructions for calculating, by the computing device, a measure of the adjustable socio-economic model's predictive quality, and instructions for displaying, by the user interface, the adjustable socio-economic model's predictive quality to the user.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the invention. Moreover, in the drawings, like reference numerals designate corresponding parts throughout the several views.

FIG. 1 is a schematic diagram of an example network environment in which an example socio-economic model system, in accordance with an embodiment.

FIG. 2 is diagram of socio-economic categories and subcategories, which may be used when generating a socio-economic model.

FIG. 3 is an embodiment of a data structure of socio-economic categories and select socio-economic factors in accordance with an embodiment.

FIG. 4 is a flow diagram of an example process for an example socio-economic model in accordance with an embodiment.

FIG. 5 is a schematic diagram that illustrates the components of the socio-economic model in accordance with an embodiment.

FIG. 6A is a diagram of a representative graphical user interface in which the user is presented with alternative socio-economic categories and tasks.

FIG. 6B is a diagram of a representative graphical user interface in which the user is presented with alternative socio-economic models and predictiveness measures.

FIG. 7 is a diagram of a representative graphical user interface in which the user is presented with a non-adherence risk record related to a task assigned to an individual.

### DETAILED DESCRIPTION OF EMBODIMENTS

It would be desirable to identify patients more likely to be noncompliant and provide timely interventions. It is believed that socio-economic factors may account for non-adherence, but heretofore there has been no efficient means to analyze this socio-economic data to predict non-adherence. The embodiments described herein seeks to overcome one or more of the deficiencies noted above. Disclosed herein are certain embodiments of a socio-economic system and method 10 that visually presents non-adherence risk based on socio-economic determinates of health. The risk score can be subsequently used for predicting non-adherence risk in an effort to enhance healthcare outcomes.

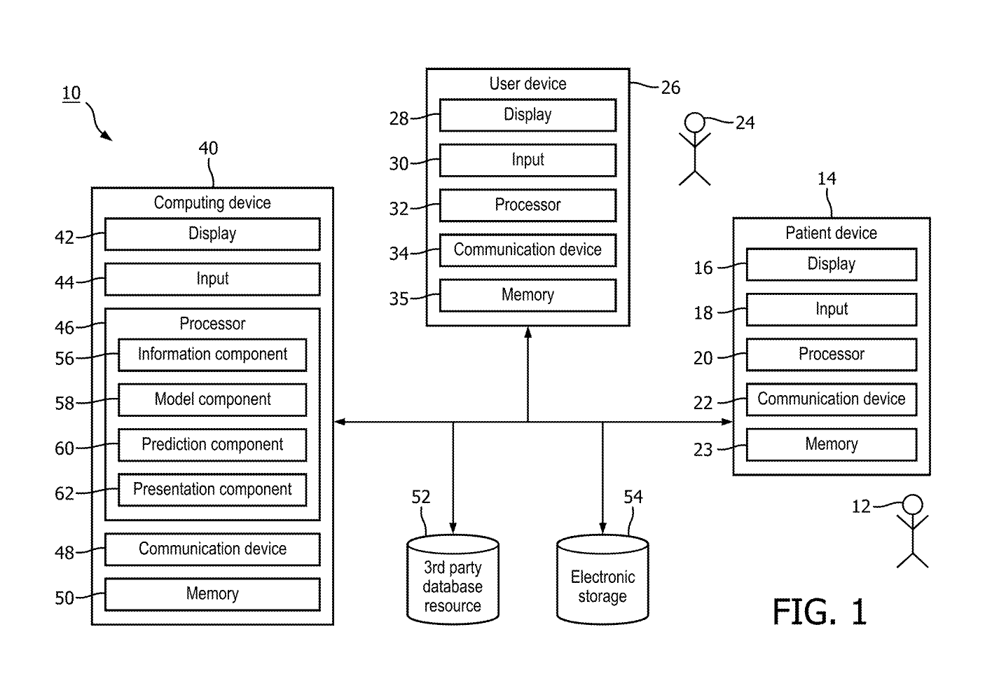

The socio-economic system and method 10 provides a measure of a variety of socio-economic factors, which have been found to correlate with non-adherence risk. Non-adherence is viewed as one of the leading causal factors leading to therapeutic failures. With reference to FIG. 1, the system 10 may be accessed by an individual 12 such as a patient who interacts with the system 10 via a patient device 14. The patient device 14 may be any of a plurality of devices with computing capability such as a desktop computer, portable computer, tablet or smartphone. The patient device 14 includes a display 16, an input 18, a processor 20, a communication device 22, and memory 23. As is well-known in the art, the display 16 may present a graphical user interface with menus, tables, icons, and the like to permit interaction with the system 10. The input 18 may be any of a variety of input devices such as a keyboard, touchscreen, computer mouse, trackball, voice recognition and the like capable of allowing the individual 12 to interact with the graphical user interface presented on the display 16. The communication device 22 is any communication device capable of connecting the patient device 14 to the system 10 such as Ethernet, Wi-Fi, Bluetooth, GSM, CDMA or the like used alone or in combination to facilitate communications. The memory 23 may be volatile, non-volatile or both. Among other things, the patient device 14 may be used by the patient, or individual, 12 to communicate self-reported health status. For instance, the patient may be asked to report whether they feel bad, good, or excellent. Alternatively, the patient may be able to report their health status on a given scale with numeric values between 0-10. Of course, one of ordinary skill in the art can appreciate that a variety of self-reporting schemes can be used with a variety of scales.

Similarly, a user 24 may be physician, nurse, administrator, or other caregiver. The user 24 may access the system 10 via a user device 26. As with the patient device 14, user device 26 also includes a display 28, input 30, processor 32, a communication device 34, and a memory 35. As is well-known in the art, the display 28 may present a graphical user interface with menus, tables, icons, and the like to permit interaction with the system. The input 30 may be any of a variety of input devices such as a keyboard, touchscreen, computer mouse, trackball, voice recognition, and the like capable of allowing the user to interact with the graphical user interface presented on the display 28. The communications device 34 may be any communication device capable of connecting the user device 26 to the system 10 such as Ethernet, Wi-Fi, Bluetooth, GSM, CDMA, or the like.

The system also includes a computing device, or computer, 40 that includes a display 42, input 44, processor 46, a communications device 48, and memory 50. As is well-known in the art, the display 42 may present a graphical user interface with menus, tables, icons, and the like to permit interaction with the system 10. The input 44 may be any of a variety of input devices such as a keyboard, touchscreen, computer mouse, trackball, voice recognition, and the like capable of allowing the user to interact with the graphical user interface presented on the display 42. The communication device 48 may be any communication device capable of connecting the computer 40 to the system 10 such as Ethernet, Wi-Fi Bluetooth, GSM, CDMA, or the like.

The system 10 also includes access to third party database resources 52, which may be internal databases or external resources from third party providers. For instance, clinical information regarding individuals 12 may be retrieved from an electronic medical records (EMR) database from providers such as Epic, Inc. or Cerner, Inc. This data includes a care plan and/or task information for each patient. For instance, the tasks may include, but are not limited to, various items related to health outcomes such as office visits, required lab tests, imaging appointments, exercise activities, medication dosing schedule, blood pressure measurements, weight measurements, and the like. Non-compliance or non-adherence to care plan tasks has been found to lead to many therapeutic failures and socio-economic factors are deemed to cause the non-compliance, but heretofore in the art there is no effective means to predict task non-adherence or determine the underlying causal factors. Now, socio-economic data is available from providers such as CentraForce, Inc. or other third party database resources 52.

The system may also include electronic storage 54, which may include private information regarding individuals collected by a healthcare provider. In addition, the electronic storage 54 may include a table, which associates a task with an associated importance index. As used herein, the task may be any task that the individual 12 has been assigned, or is expected to perform, such as taking various medications, performing exercises, a physical therapy routine, attend an office visit, have a test taken, blood pressure measurement, weight measurement, or have a medical procedure. Each task in the table has an associated importance index. For example, some tasks may be more important than others regarding the individuals 12 health outcome. Missing a single weight measurement may not be very significant and given a low index. On the other hand, not taking a particular medication or missing a surgical appointment on the other hand could be more critical and given a higher index.

The database resource 52 and electronic storage 54 may be co-located within the first user device 14, the second user device 26, the computing device 40, or remote from devices 14, 26, and 40 (as depicted). Ideally, given the size of the databases contained in either the database resources or electronic storage, these databases 52, 54 are preferably contained in cloud-based data storage such as that provided by SalesForce.com, Inc, Amazon Web Services, Inc. or other similar providers.

The processor 46 of computing device 40 includes an information component 56, a model component 58, a prediction component 60, and a presentation component 62. The information component 56 is configured to process information about individuals 12 such as claims data, clinical data or socio-economic data received from the databases 52, 54. The model component 58 is configured to define and train a socio-economic model, which correlates the selected socio-economic factors as explanatory variables with a non-adherence risk score as a response variable via weighting factors associated with each socio-economic factor. Lastly, the presentation component 62 displays the output of the model to the user 24 on display 16 and inquires whether the user 24 wishes to accept the model or make adjustments and retrain the model. The user provides these responses via the input 18.

While the network environment of the system depicted in FIG. 1 contemplates that the patient device 14, the user device 26 and the computing device 40 may be remote from one another, one of ordinary skill in the art can readily appreciate that one or more of these components could be co-located in a single device or that further segregation of components could be made.

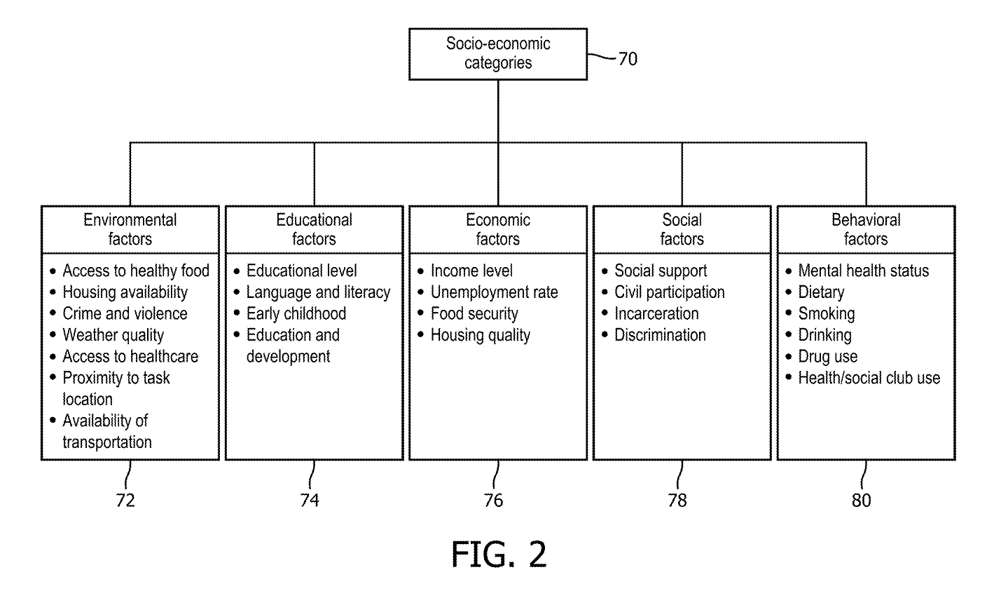

As seen in FIG. 2, there are multiple socio-economic factors that can be taken into account. FIG. 2 provides a non-exhaustive example of some of the more common socio-economic categories and factors. Here, the socio-economic categories 70 are represented as 5 categories: environmental 72, educational 74, economic 76, social 78, and behavioral 80 categories. In turn, each of these categories 72-80 may include various related factors. For instance, as depicted, the environmental category 72 includes factors such as access to healthy food, housing availability, crime and violence rate, weather quality, access to healthcare, proximity to task location, availability of transportation. The education category 74 includes factors such as education level, language and literacy, early childhood development, and education and development. The economic category 76 includes factors such as income level, unemployment rate, food security, and housing quality. The social category 78 includes factors such as social support network, civil participation, incarceration, and discrimination. Lastly, the behavioral category 80 includes factors such as smoking habit, drinking, drug use, dietary status, health/social club use, and mental health status. While not exhaustive, FIG. 2 provides a representative example of the socio-economic factors that are generally seen as impacting an individual's non-adherence risk. Currently, caregivers have little insight into these factors and are thus unable to consider the entire picture when seeking to treat a patient often leading to costly sub-optimal outcomes when their patient becomes non-adherent. Heretofore, there has not been an effective tool for analyzing socio-economic factors which can correlate the impact that these factors have on non-adherence, or more importantly capable of doing a root-cause analysis to determine what socio-economic factors are leading to the non-adherence risk.

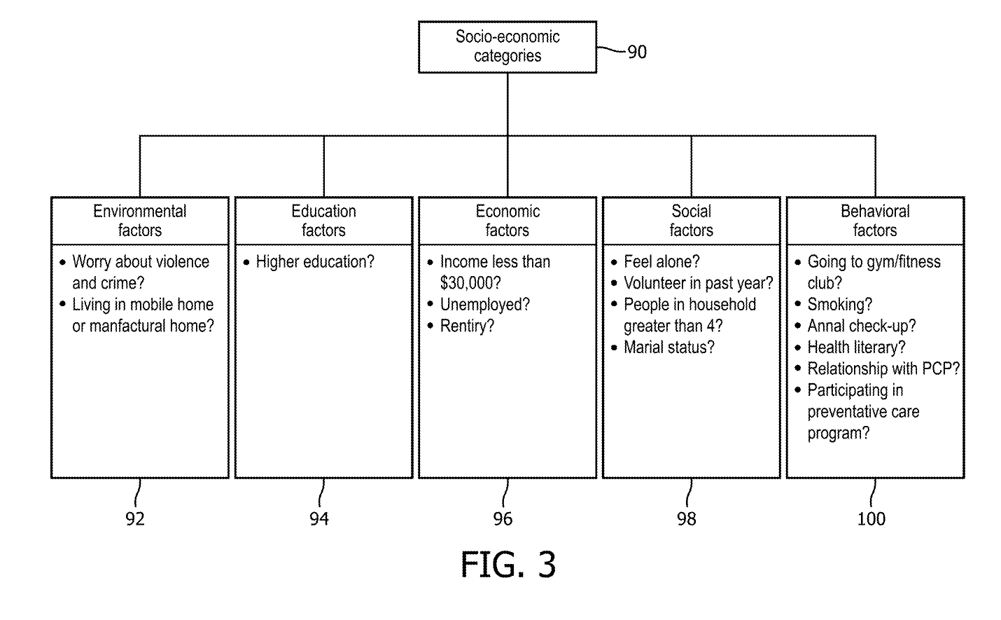

While having access to all of the information depicted on FIG. 2 would be ideal, much of this data has proven difficult to collect and process. As such, simplifications can made to focus on key factors for which sufficient data exists, which is adequately predictive. One embodiment, which seeks to simplify the factors utilized, is depicted in FIG. 3. Here, the socio-economic categories 90 have been simplified to include environmental 92, educational 94, economic 96, social 98, and behavioral 100 factors. In this simplified embodiment, the environmental category 92 includes two factors: does the individual worry about crime and violence, and are you living in a mobile home, manufactured home or other temporary structure. As for the educational category, the only factor is whether the individual has attained higher education or not. The economic category 96 includes factors such as whether the individual's income level is higher or lower than a threshold (e.g. $30,000), whether the individual is unemployed or not, and whether the individual has stable housing or not (e.g. renting versus owning). The social category 98 includes factors such as whether the individual feels lonely, do they volunteer or participate in community events, whether the number of people in the household is greater than 4, and marital status (e.g., married, single, divorced). Lastly, the behavioral category 100 includes factors such current gym participation, smoking, annual check-up, Relationship with primary care physician, and participation in preventative health activities. While less comprehensive than the factors listed in FIG. 2, these factors are more readily available and capable of being processed in a quick and efficient manner via data that is readily available and can lead to satisfactory results. Of course, one of ordinary skill in the art can readily appreciate this example is merely demonstrative, a multitude of different factors could be added, deleted, or substituted without departing from the scope of the present embodiments.

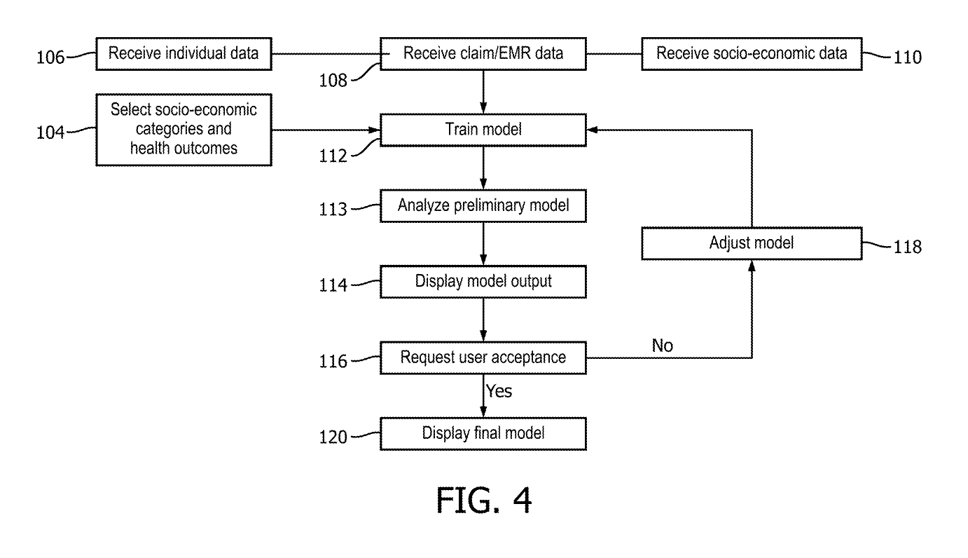

With reference to FIGS. 1 and 4, the system 10 permits the user 24 to select which socio-economic categories 92-100 should be used to calculate the model as described in co-pending patent application (U.S. Patent Application No. 62/587,921 filed Nov. 17, 2017), which is incorporated herein by reference. The Information component 56 receives individual data 106, claims or electronic medical records (EMR) data 108, and socio-economic data 110. The individual data 106 includes data received from the patient device 14, which may include compliance, or non-compliance, data related to the individual's 12 adherence to the assigned care plan tasks. As, will be described below, the model component 58 uses the selected socio-economic categories to select the associated socio-economic factors for each socio-economic category to calculate a model which correlates the socio-economic factors with a non-adherence risk metric or score as the output or response variable 126.

The information component 56 also receives data from claims or EMR databases 108. Again, these databases may come from external providers stored on 3rd party database resources 52. Or, the data may be private in nature and stored on electronic storage 54. In a preferred embodiment, the inventors contemplate that information regarding the patient's 12 age, ethnicity, marital status, and zip code or address can be extracted from the database 52, 54. This data can be used as inputs to extract the appropriate data from the socio-economic database contained on a third party database resource 52, or electronic storage 54. In addition, the claims or EMR database may also include the individual's care plan including expected or assigned tasks for the individual 12.

The information component 56 also receives data from socio-economic databases 110, which is extracted based on the EMR and/or claims data received from claims or EMR databases 108 noted above. The socio-economic data 110 may be located on external data resources 52 such as from a public provider like CentraForce. Alternatively, the socio-economic data 104 may be a private database stored in electronic storage 54, which may be resident with computing device 40, or remote from computing device 40.

In step 104, the computing device 40 uses the data 106, 108, and 110 to select the relevant data related to the selected factors 92-100 of the categories 90. For example, the system will use the individual's zipcode or address to extract the relevant socio-economic factors associated with the patient's zipcode or address while excluding the factors that are not as predictive of non-adherence. Next, in step 112, the system trains a model 122 using the selected data to provide a non-adherence risk score 126. Machine learning techniques are applied to create a model 122, which fits the data and is predictive of non-adherence. The model includes socio-economic factors 134 and associated weighting factors 136. For example, the computing device 40 may calculate a model 122 such as:

SES=(Af)*(Age)+(Uf)*Unemployment+(AQf)*Air quality, where

SES is the Socio-economic or non-adherence risk score 126;

Age is the age of the patient 134;

Unemployment represents the unemployment status of the patient 134;

Air quality represents the air quality the patient is exposed to 134;

Af is the Age weighting factor 136;

Uf is the unemployment factor 136; and

AQf is the Air Quality factor 136.

In one embodiment, linear regression may be employed as the machine learning technique to develop the socio-economic model 122. However, a variety of other techniques can be employed as well such as Bayesian linear regression, least-angle regression, theil-sen estimator, Lasso, Ridge, or even polynomial regression techniques could be employed.

In step 113, the prediction component 60 calculates the predictive quality of the model 122 generated by the model component 58. In a preferred embodiment this can be accomplished by performing a Root-Mean-Square Error (RMSE) and R-Square R² values for the model 122. These values are well-known in the art and would provide the user with a ready metric indicating the quality of the model 122. Of course, a variety of other techniques could be used to assess the predictive quality of the model 122 such as average mean variance, median mean variance, and average absolute deviation.

The presentation component 62 displays the model generated by the model component 58 and the predictive quality of the model generated by the prediction component 60 for the user 24 on display 28 in step 114. In step 116, the user 24 is given the option to accept or reject the model 122. If the user 24 accepts the model, the user 24 is given the option to display and save the model 120. If the user rejects the model, the user is allowed to adjust the model in step 118 via input 30 of the user device 26.

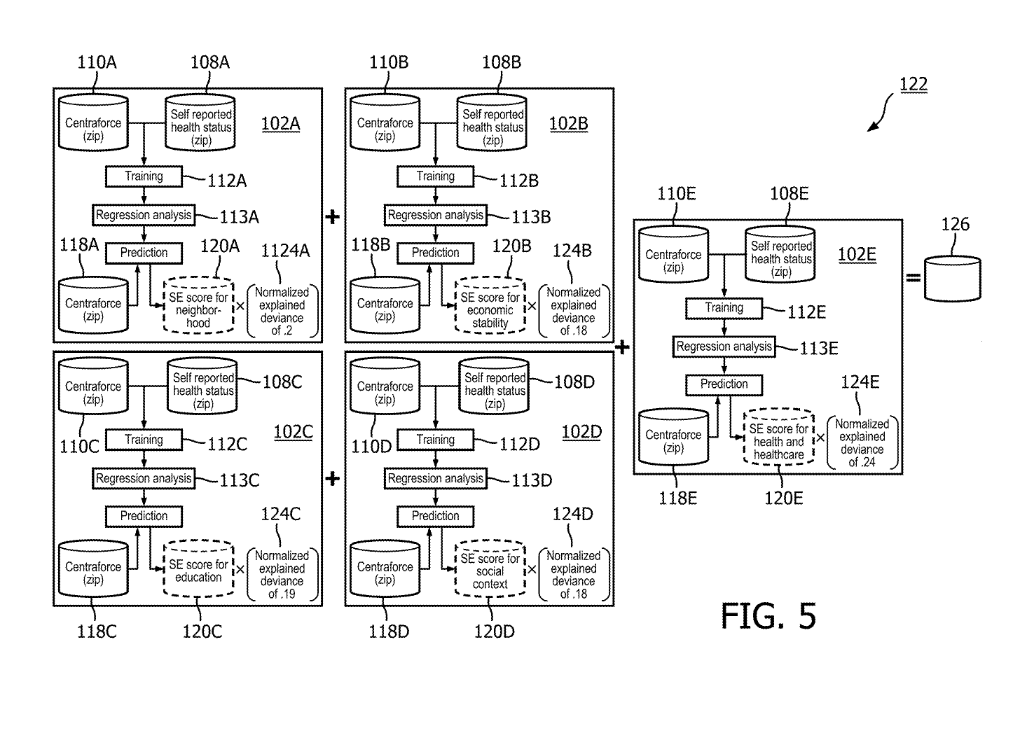

While the preceding description is one demonstrative embodiment wherein the model is generated with each socio-economic category processed together to generate a singular, holistic socio-economic model, as seen in FIG. 5, it is also possible to generate the socio-economic model 122 based on individual socio-economic models for each category 92-100. This allows for each category to be individually calculated and weighted via secondary weighting factors. The weighted categories can then be combined, or summed, into a combined socio-economic model. FIG. 5 shows an embodiment of this configuration wherein like features are given the same reference as described above with respect to FIG. 4 wherein the Environment category references are appended with the letter A, the Economic category are appended with the letter B, the Educational category references are appended with the letter C, the Social category references are appended with the letter D, and the Behavioral category references are appended with the letter E. Each category 102A-102E includes a secondary weighting component 124A-124E. The results are then summed to create the socio-economic model 122.

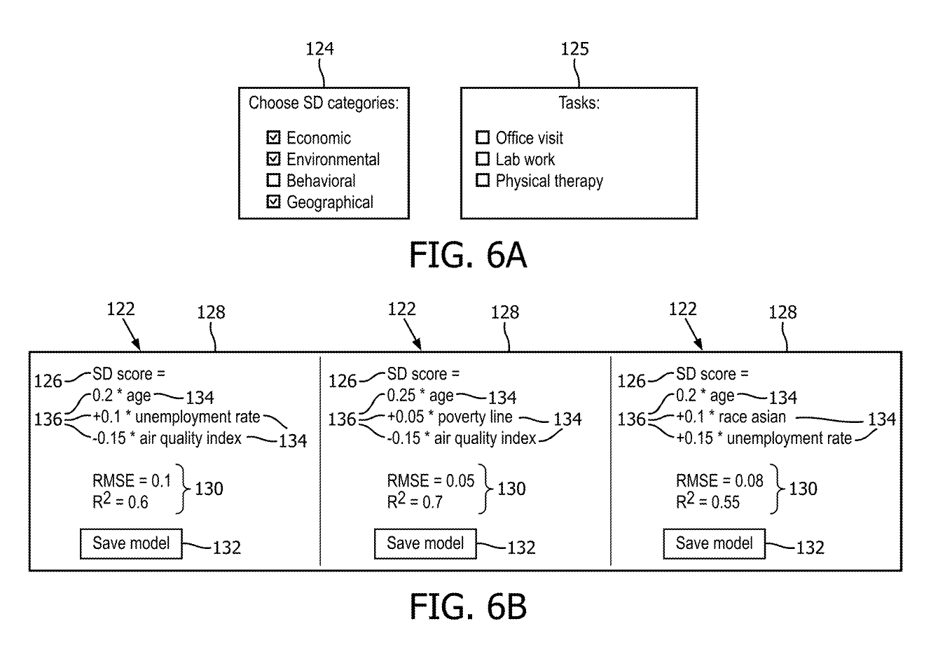

With reference to FIG. 6A, a demonstrative graphical user interface is shown which would be presented on the display 28 of the user device 26. Here, the user 24 can select which socio-economic categories should be included when generating the socio-economic model 122. For instance in the example shown, the economic 96, environmental 92, and social 98 categories have been selected by the user 24 represented by the checks in the displayed boxes. For this model 122, the user has concluded that the behavioral category is not relevant to the model. This could be for experimental purposes or it could be that the user 24 has concluded that behavioral factors are irrelevant, or even worse, may detune the model 122 and decreased predictiveness.

FIG. 6A further shows that the system 10 provides a user interface that allows the user 24 to select the socio-economic categories 124 and to select the desired task 125 for non-adherence risk analysis. In this embodiment, the output can be generated with respect to a particular patient 12 to provide an index representing an assessment of the selected patients 12 non-adherence risk for a selected task. This can be accomplished by inputting into the model the individual's data for the socio economic factors 134. This data can be obtained from the received individual data 106, claim/EMR data 108, or socio-economic data 110. Alternatively, the output could be used retroactively to explore why a patient 12 was non-adherent. In another embodiment, the model could be applied to multiple individuals in a patient population to risk assess multiple patients within a population. This would allow the user 24 to rank risk and triage, or prioritize, patient's 12 based on their determined non-adherence risk. The user 24 could determine that the highest at risk individuals will require additional socio-economic support (e.g., subsidies, at-home nursing, transportation, emotional support, etc.) as compared to individuals with low socio-economic risk scores, thus optimizing care and steering resources towards the patients with the greatest need.

FIG. 6B shows a representative user interface presented on display 28 with the model and quality metrics displayed of step 114. For each of the selected categories 92-100, the model 122 is displayed showing the socio-economic factors 134 including their associated weighting factors 136. In addition, this depicted graphical user interface also provides exemplary RMSE and R² values 130 to provide the user 24 with an estimate of the model's 122 predictive quality. The user 24 can accept each of these models as final and press the save button 132, or the user 124 can make adjustments to the adjustable model 128 in step 118 and resubmit the model to the model component 58 to recalculate a new version of the model in step 112.

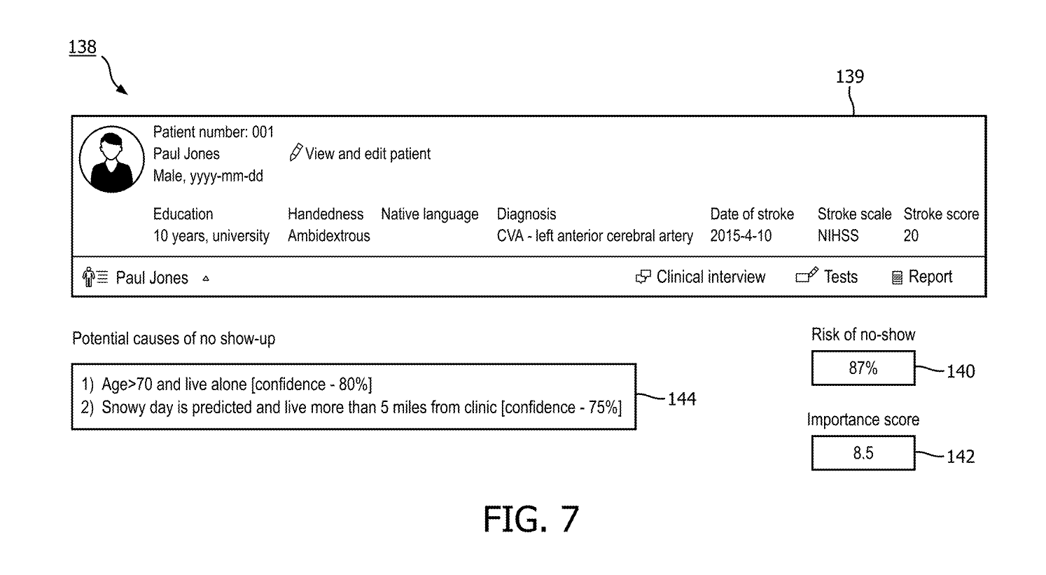

As shown in FIG. 7, once the user has selected a final model 132, in step 120, the display 28 provides the user 24 with a user interface showing the output of the system 10 with data calculated for a selected individual 12. The user interface displays the individual's non-adherence risk record 138 which includes the patient task info 139, the non-adherence risk index 140, the task importance index 142, and the root-cause 144 leading to the risk. In this way, the user 24 is presented with a measure of the patient's non-adherence risk, the importance of the task as well as the underlying root-causes related to the selected task. Collectively, this information provides the user 24 with the necessary information to detect issues and improve patient care.

Non-adherence risk is an important piece of information, which has previously been unavailable to users 24. It allows the user 12 to determine how likely it is that a patient will not be adherent to a particular task. In addition, the embodiments presented herein also provide a measure of the importance of the task. Even if there is a risk of non-adherence, not all risks warrant intervention based on their importance. The task importance index helps the user 24 determine severity or importance of the risk. Some tasks may be less likely to cause poor health outcomes and others may be more likely to result in poor health outcomes. For instance, taking a blood pressure reading on a daily basis may be desirable but missing a few readings will not lead to an immediate health crisis. However, not taking critical medication on a daily basis or missing a medical procedure could lead to an immediate health crisis. The importance can be determined by referencing the table that correlates tasks with the task importance index. The display shown in FIG. 7 provides the user 12 with a measure of the task criticality, or importance, 142.

Lastly, the user interface also displays the results of a root-cause analysis performed on the model 122. Another advantage of the embodiments presented herein is that they provide a measure of what is causing the non-adherence risk so that users may be able to determine what intervention might be needed to correct the non-adherence. The root-cause analysis can be determined in a variety of manners. Preferably, the root-cause analysis can be performed by using an Apriori algorithm. As is well-known in the art, an Apriori algorithm can be used to identify the strength of the association, or correlation, between the explanatory variables, which in this application are socio-economic factors, and the selected response variable, which in this application is a selected measure of non-adherence to a selected task, to determine the relationship between the two. Of course, a variety of other association rule learning techniques could be employed such as the Eclat algorithm, FP-growth algorithm, and the like.

Simplistically, the root-cause factors could also be determined by ranking socio-economic factors along with their associated weighting. The weighting factor provides a measure of the importance of the socio-economic factor within the population used to generate the model, and the socio-economic factor provides a measure of the significance of that particular socio-economic factor to the selected patient 12. The multiple of the socio-economic factor and its associated weighting may be ranked from largest to smallest, with larger multiples being more impactful to the non-adherence risk index.

Once the root-cause analysis has been performed as described above, the user interface of the display 28 provides the results of a root-cause analysis 144 which identifies the socio-economic factors having the strongest impact on the risk index 140. The results 144 may include all of the socio-economic factors impacting the non-adherence risk. Alternatively, the results 144 may be a shortened list of the top socio-economic factors, as shown.

As seen in FIG. 7, the selected individual information record 138 is related to an upcoming task. The patient's 12 task related info 139 is displayed. As described above, the model 122 was created and a non-adherence risk index 140 of 87% was calculated for the individual 12 predicting that there is an 87% chance that the patient 12 will not adhere to the task. The user 24 can then review the task importance index 142. In this example, the importance index 142 is 8.5 on a scale of 1-10, which indicates that the importance of the task is on the higher end of the scale. So taken together, the system indicates that the individual 12 in this example has a relatively high risk of being non-adherent and the task is relatively important, which indicates an intervention may be warranted. However, the user 24 may not readily understand why the individual 12 is likely to be non-adherent or what to do about it. A novel aspect of the system is that the user 24 can review the root cause results 144 displayed on the graphical user interface and see which the socio-economic factors are elevating the non-adherence risk. Here the root-cause results indicate that the weather is predicted to be poor, the patient lives more than 5 miles from the office location, and this patient is greater than 70 years old and is single. Taken together, there is a higher probability that this patient would have to make this trip on their own and that it might be a difficult trip due to their age and weather putting compliance at risk. This provides the user 24 with a better view into the individual's 12 socio-economic status and provides insight into why the individual is unlikely to be adherent. To resolve this problem, the caregiver may suggest that a transportation service be employed or that a family member should be contacted to ask if they could help. While this may result in a modest increase in time or money, it may in actuality increase the patient's adherence resulting in a better long-term outcome for the patient and reduced cost.

The embodiments described herein can also be used to enhance care if provided to a physician, nurse or other caregiver. While the embodiments have been described prospectively, it could also be used retrospectively to determine why a particular has become non-adherent and missed an appointment to determine the root-cause. Historically, physicians had little insight into socio-economic components, but simply providing the caregiver with all the data related to an individual under their care may not solve the problem even if the individual would be willing to provide it given the amount of data that would need to be analyzed. Most caregivers are under extreme time pressures and are not data scientists. Finding this data, analyzing it and determining the correlations between socio-economic factors with non-adherence is simply too time consuming and complicated to perform. The embodiments described herein could provide caregivers with a ready tool to provide quick and meaningful insight regarding a patient's socio-economic factors that may impact non-adherence so they can focus additional services on the patients that are most likely in need of additional support and avoid providing services to patients that do not need additional support with the goal of increasing adherence, improved outcomes, and minimizing expense. In addition, if the socio-economic score is high due to behavioral factors. The user may, as part of their care plan, prescribe social services such as a counseling, a support group, etc. Or, if the patient is being discharged, the discharging physician may consider whether additional community support or at-home care is justified in an effort to avoid the cost associated with readmission. So, in a way, the system could provide socio-economic decision support mirroring clinical decision support which is ubiquitous in the industry.

In general, the embodiments described herein provide caregivers a tool that helps analyze a wide variety of information available about a patient and provide information related to the problem of patient non-adherence. This includes the use of socioeconomic data that has been found to correlate with the level of patient adherence. Accordingly, the embodiments presented herein provide a technological solution to the problem of nonadherence.

Although the description identifies or describes specifics of one or more embodiments, such specifics are not necessarily part of every embodiment, nor are all various stated advantages associated with a single embodiment. On the contrary, the intent is to cover all alternatives, modifications and equivalents included within the scope of the socio-economic system and method as defined by the appended claims. Further, it should be appreciated in the context of the present disclosure that the claims are not necessarily limited to the particular embodiments set out in the description.

Note that reference to thresholds refers to minimum triggers for certain conditions for actions to commence. The thresholds may be based on historical or experimental data, or based on the expertise of a user and/or context. In some embodiments, a threshold may be established based on a combination of experience and context.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

At least the following is claimed:

1. A system for producing an adjustable socio-economic adherence model, comprising:
a database containing socio-economic data which classifies socio-economic factors into socio-economic categories and containing electronic medical record (EMR) data for a plurality of patients;
a user interface displayed on a display device to a user, wherein the user interface is configured to:
display socio-economic categories to the user; and
receive user inputs selecting one or more socio-economic categories;
a computing device configured to produce the adjustable socio-economic adherence model, wherein the computing device is configured to:
extract training patient data from the EMR data including patient task adherence data;
extract socio-economic training data for the patients from the socio-economic data based upon socio-economic data and the user selected socio-economic categories; and
train the adjustable socio-economic model using the extracted patient data and extracted socio-economic training data, wherein the adjustable socio-economic model produces a non-adherence risk score related to the patient task.

2. The system of claim 1, wherein the socio-economic categories include an environmental category, an educational category, an economic category, a social category, and a behavior category.

3. The system of claim 1, wherein
the user interface is further configured to
present individuals with upcoming tasks; and
receive a user selection of an individual and a task assigned to the individual; and
the computing device is further configured to calculate a non-adherence risk score for the selected individual and selected task using the adjustable socio-economic model.

4. The system of claim 3, wherein
the system includes a table that correlates tasks with a task importance index,
the non-adherence risk score is further based on the task importance index,
the computing device is further configured to train the adjustable socio-economic model to calculate a root-cause related to the non-adherence risk, and
the user interface is further configured to display the task non-adherence score, the task importance index, and the root-cause related to the non-adherence for a selected individual.

5. The system of claim 1, wherein the system includes a table that correlates tasks with a task importance index and wherein the non-adherence risk score is further based on the task importance index.

6. The system of claim 1, wherein the computing device is further configured to train the adjustable socio-economic model to calculate a root-cause related to the non-adherence risk.

7. The system of claim 1, wherein training the adjustable socio-economic model comprises training a sub-model for each socio-economic category and wherein the non-adherence risk score is calculated as a weighted sum of each of the sub-models.

8. The system of claim 1, wherein
the computing device is further configured to calculate a measure of the adjustable socio-economic model's predictive quality, and
the user interface is further configured to display the adjustable socio-economic model's predictive quality to the user.

9. A method of producing an adjustable socio-economic adherence model, comprising:
displaying, by a user interface, socio-economic categories to a user;
receiving, by the user interface, user inputs selecting one or more socio-economic categories;
extracting, by a computing device, training patient data from emergency electronic medical record (EMR) data in a database including patient task adherence data;
extracting, by the computing device, socio-economic training data for the patients from socio-economic data in the database based upon the user selected socio-economic categories; and
training, by the computing device, the adjustable socio-economic model using the extracted patient data and extracted socio-economic training data, wherein the adjustable socio-economic model produces a non-adherence risk score related to the patient task.

10. The method of claim 9, wherein the socio-economic categories include an environmental category, an educational category, an economic category, a social category, and a behavior category.

11. The method of claim 9, further comprising:
presenting, by the user interface, individuals with upcoming tasks;
receiving, by the user interface, a user selection of an individual and a task assigned to the individual; and
calculating, by the computing device, a non-adherence risk score for the selected individual and selected task using the adjustable socio-economic model.

12. The method of claim 11, further comprising:
training, by the computing device, the adjustable socio-economic model to calculate a root-cause related to the non-adherence risk; and
displaying, by the user interface, a task non-adherence score, a task importance index, and the root-cause related to the non-adherence for a selected individual,
wherein the system includes a table that correlates tasks with a task importance index, and
wherein the non-adherence risk score is further based on the task importance index,

13. The method of claim 9, wherein the system includes a table that correlates tasks with a task importance index and wherein the non-adherence risk score is further based on the task importance index.

14. The method of claim 9, further comprising training, by the computing device, the adjustable socio-economic model to calculate a root-cause related to the non-adherence risk.

15. The method of claim 9, wherein training the adjustable socio-economic model further comprises training a sub-model for each socio-economic category and wherein the non-adherence risk score is calculated as a weighted sum of each of the sub-models.

16. The method of claim 9, further comprising:
calculating, by the computing device, a measure of the adjustable socio-economic model's predictive quality, and
displaying, by the user interface, the adjustable socio-economic model's predictive quality to the user.

17. A non-transitory machine-readable storage medium encoded with instructions for producing an adjustable socio-economic adherence model, comprising:
instructions for displaying, by a user interface, socio-economic categories to a user;
instructions for receiving, by the user interface, user inputs selecting one or more socio-economic categories;
instructions for extracting, by a computing device, training patient data from emergency electronic medical record (EMR) data in a database including patient task adherence data;
instructions for extracting, by the computing device, socio-economic training data for the patients from socio-economic data in the database based upon the user selected socio-economic categories; and
instructions for training, by the computing device, the adjustable socio-economic model using the extracted patient data and extracted socio-economic training data, wherein the adjustable socio-economic model produces a non-adherence risk score related to the patient task.

18. The me non-transitory machine-readable storage medium of claim 17, wherein the socio-economic categories include an environmental category, an educational category, an economic category, a social category, and a behavior category.

19. The non-transitory machine-readable storage medium of claim 17, further comprising:
instructions for presenting, by the user interface, individuals with upcoming tasks;
instructions for receiving, by the user interface, a user selection of an individual and a task assigned to the individual; and
instructions for calculating, by the computing device, a non-adherence risk score for the selected individual and selected task using the adjustable socio-economic model.

20. The non-transitory machine-readable storage medium of claim 19, further comprising:
instructions for training, by the computing device, the adjustable socio-economic model to calculate a root-cause related to the non-adherence risk; and
instructions for displaying, by the user interface, a task non-adherence score, a task importance index, and the root-cause related to the non-adherence for a selected individual,
wherein the system includes a table that correlates tasks with a task importance index, and
wherein the non-adherence risk score is further based on the task importance index,
